(19) United States
(12) Patent Application Publication
(10) Pub. No.: US 2025/0135208 A1
(43) Pub. Date: May 1, 2025

(54) Use of N-let Pulses in a Deep Brain Stimulation System to Selectively Treat Symptoms

(71) Applicant: Boston Scientific Neuromodulation Corporation, Valencia, CA (US)

(72) Inventors: Que Doan, West Hills, CA (US); Michael Moffitt, Solon, OH (US)

(21) Appl. No.: 18/931,821

(22) Filed: Oct. 30, 2024

Related U.S. Application Data
(60) Provisional application No. 63/594,855, filed on Oct. 31, 2023.

(51) Int. Cl. A61N 1/36; A61N 1/372; A61N 1/375

(52) U.S. Cl. CPC A61N 1/36139; A61N 1/36132; A61N 1/36171; A61N 1/36178; A61N 1/36182; A61N 1/37211; A61N 1/3756

(57) ABSTRACT

Stimulation using N-lets (e.g., doublets, triplets, etc.) in a Deep Brain Stimulation system are disclosed. Pulses are issued in packets of N-lets, resulting in a waveform with both high and low frequency aspects, which are independently adjustable to treat different symptoms. For example, adjustment of higher frequency aspects can be used to treat tremor, while adjustment of lower frequency aspects can be used to treat freezing gait. Such adjustments may be made manually via a graphical user interface, or automatically using an algorithm.

### CROSS REFERENCE TO RELATED APPLICATIONS

This is a non-provisional application of U.S. provisional application Ser. No. 63/594,855, filed Oct. 31, 2023, which is incorporated herein by reference in its entirety, and to which priority is claimed.

### FIELD OF THE INVENTION

This application relates to Implantable Stimulator Devices (ISD), and more specifically to stimulation strategies for treating the particular symptoms in Deep Brain Stimulation (DBS) patients.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system, such as that disclosed in U.S. Patent Application Publication 2020/0001091, which is incorporated herein by reference. However, the present invention may find applicability with any implantable neurostimulator device system, including Spinal Cord Stimulation (SCS) systems, Vagus Nerve Stimulation (VNS) system, Sacral Nerve Stimulation (SNS) systems, Peripheral Nerve Stimulation (PNS) systems, and the like.

A DBS system typically includes an Implantable Pulse Generator (IPG) 10 shown in FIG. 1. The IPG 10 includes a biocompatible device case 12 that holds the circuitry and a battery 14 for providing power for the IPG to function, although the IPG 10 can also lack a battery and can be wirelessly powered by an external source. The IPG 10 is coupled to tissue-stimulating electrodes 16 via one or more electrode leads forming an electrode array 17. The electrodes as shown comprise ring electrodes, but split-ring electrodes can also be used that allow stimulation to be provided at different circumferential directions around the lead, as discussed further for example in U.S. Patent Application Publication 2022/0257950.

Lead wires 20 within the leads are coupled to the electrodes 16 and to proximal contacts 21 insertable into lead connectors 22 fixed in a header 23 on the IPG 10, which header can comprise an epoxy for example. Alternatively, the proximal contacts 21 may connect to lead extensions (not shown) which are in turn inserted into the lead connectors 22. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below. In the IPG 10 illustrated in FIG. 1, there are four percutaneous leads 18, and thus the header 23 may include a 2×2 array of eight-electrode lead connectors 22. However, the type and number of leads, and the number of electrodes on each, in an IPG is application specific and therefore can vary. The conductive case 12 can also comprise an electrode (Ec).

In a DBS application, as is useful in the treatment of tremor in Parkinson's disease for example, the IPG 10 is typically implanted under the skin below the patient's clavicle (collarbone). Leads including the electrodes 16 (perhaps as extended by lead extensions, not shown) are tunneled through and under the neck and the scalp, with the electrodes 16 implanted through holes drilled in the skull and positioned for example in the subthalamic nucleus (STN) and the pedunculopontine nucleus (PPN) in each brain hemisphere. The IPG 10 can also be implanted underneath the scalp closer to the location of the electrodes' implantation, as disclosed for example in U.S. Pat. No. 10,576,292. The electrode array 17 can be integrated with and permanently connected to the IPG 10 in other solutions.

IPG 10 can include an antenna 27a allowing it to communicate bi-directionally with a number of external devices and systems discussed subsequently. Antenna 27a as shown comprises a conductive coil within the case 12, although the coil antenna 27a can also appear in the header 23. When antenna 27a is configured as a coil, communication with external systems preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like. Although not shown, the IPG 10 may include an additional coil to receive wireless power from an external source, or to charge the IPG's battery 14.

IPG 10 may include control circuitry 29, which can comprise any number of devices such as one or more microprocessors, microcomputers, microcontrollers, FPGAs, DSPs, other digital logic structures, etc., which are capable of executing programs in a computing device. Such control circuitry 29 may contain or be coupled with memory which can store software or firmware for operating the IPG 10. Control circuitry 29 may include, or be in communication with, stimulation circuitry 28 in the IPG 10, as discussed further below.

Stimulation in IPG 10 is typically provided by pulses each of which may include a number of phases as shown in the example of FIGS. 2A and 2B. In the examples shown, such stimulation is monopolar, meaning that a current is provided using a single pole in the electrode array 17. In monopolar stimulation, the case electrode Ec 12 is used as a current return and is driven with the opposite polarity. The single pole formed on the electrode lead can be formed by selection of more than one lead-based electrode with the same polarity. For example, a positive current provided simultaneously at electrodes E1 and E2 would create a single anodic pole in the electrode array that is positioned between those electrodes. See, e.g., U.S. Pat. No. 10,881,859. Although not shown, stimulation can also be multipolar, in which at least one anodic pole and at least one cathodic pole is formed in the electrode array 17. For example, simultaneously providing a positive current at electrode E1 and a negative current at electrode E2 would create an anodic pole and a cathodic pole on the lead, thus comprising an example of bipolar stimulation. Again, each of these poles can be formed in the electrode array 17 using more than one electrode.

Stimulation parameters typically include amplitude (current I, although a voltage amplitude V can also be used); frequency (F); pulse width (PW) of the pulses or of its individual phases; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

FIG. 2A shows an example in which monopolar stimulation is provided using biphasic pulses having two actively-driven phases 30a and 30b. E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude+I to the tissue. Note that at any time the total current sunk from the tissue equals the total current sourced to the tissue. The polarity of the currents change in second phase 30b, with E1 now being driven as an anode (+I), and with the case electrode Ec being driven as a cathode (−I). As discussed above, monopolar stimulation could be provided by using more than one active electrode. For example, the cathodic current −I could be spilt between E1 and E2 (e.g., each providing 50%*−I) in phase 30a to form a cathodic pole positioned between these electrodes.

Biphasic pulses are useful because they provide active charge recovery, meaning that a current can be actively driven by the stimulation circuitry 28 during the second phase 30b to actively recover charge that was injected and stored during the first phase 30a. Such charge storage can occur on capacitances in the electrode current paths causing unwanted voltage build up, such as on the DC blocking capacitors 38 (FIG. 3) discussed subsequently. These capacitances will charge during the first phase 30a, but this charge is actively recovered during the second phase 30b. To achieve the industry standard for ideal charge recovery, the charge injected during the first phase 30a (e.g., at E1, −Q=−I*PW) preferably equals the charge injected during the second opposite-polarity phase 30b (e.g., at E1, +Q=+I*PW), meaning that the biphasic pulses is charge balanced at each active electrode. Equating this charge can occur by using a biphasic pulse whose phases 30a and 30b are symmetric—i.e., having the same amplitude I and pulse width PW (although differing in polarity), as shown in FIG. 2A. However, although not shown, charge balance can also be achieved using an asymmetric biphasic pulse in which the phases 30a and 30b have the same charge Q, but differ in their amplitudes and pulses widths.

Active charge recovery using biphasic pulses may not be perfect, because non-idealities in the circuitry (FIG. 3) used to produce the pulses may not produce the actively-driven phases 30a and 30b with exactly equal and opposite charges +Q and −Q. To the extent residual charge is left over that still needs to be recovered at the end of the second phase 30 (ΔQ), that phase can be followed by a passive charge recovery phase 30c. Passive charge recovery does not involve the active driving of a current at the electrodes, but instead essentially shorts the electrodes together to cause residual stored charge to passively equilibrate through the patient's tissue, as explained further below with reference to FIG. 3. This results in a RC decay of the current, as shown by the exponential decay during passive recovery phase 30c in FIG. 2A.

FIG. 2B shows an example in which monopolar stimulation is provided using monophasic pulses having only one actively-driven phase 30a. As before, E1 has been selected as a cathode during this phase 30a providing pulses which sink a negative current of amplitude −I from the tissue, while the case electrode Ec has been selected as an anode providing pulses which source a corresponding positive current of amplitude+I. (Again, more than one electrode may be active when forming the cathodic pole). Because charge will likely be stored on capacitances during this phase 30a that requires recovery, it is typical that the actively-driven phase 30a is followed by a passive recovery phase 30c. Because this phase 30c must recover all of the injected charge Q during phase 30a (and not just a residual remaining charge ΔQ following an active charge recovery phase 30b), the resulting passive charge recovery current during phase 30c is significantly larger as shown.

The IPG 10 as mentioned includes stimulation circuitry 28 to form prescribed stimulation such as the pulses just described at a patient's tissue, Z. FIG. 3 shows an example of stimulation circuitry 28, which includes one or more current sources (PDACs) and one or more current sinks (NDACs). These sources and sinks can comprise Digital-to-Analog converters (DACs), and respectively produce positive (sourced, anodic) and negative (sunk, cathodic) currents. In the example shown, a NDACi/PDACi pair is dedicated (hardwired) to a particular electrode node ei 39. Each electrode node ei 39 is connected to an electrode Ei 16 via a DC-blocking capacitor Ci 38, for the reasons explained below. PDACs and NDACs can also comprise voltage sources.

Proper control of the PDACs and NDACs allows any of the electrodes 16 and the case electrode Ec 12 to be actively driven to create a current (such as the pulses described earlier) through a patient's tissue, Z, hopefully with good therapeutic effect. In the example shown, and consistent with pulse phase 30a of FIGS. 2A and 2B, electrode E1 has been selected as a cathode electrode to sink current from the tissue Z, and case electrode Ec has been selected as an anode electrode to source current to the tissue Z. Thus PDACc (coupled to Ec) and NDAC1 (coupled to E1) are activated and digitally programmed to drive the desired current, I, with the correct timing (e.g., in accordance with the prescribed frequency F and pulse width PW). During another actively-driven pulse phase such as 30b (FIG. 2A), different DACs can be activated, such as PDAC1 and NDACc to reverse the polarity of the current.

During passive charge recovery phases 30c, the DACs are not active to drive current, but instead passive recovery switches 41 are closed. For example, at least switches 411 and 41c associated with previously-active electrodes E1 and Ec can be closed to connect the capacitances at these electrodes to a common node (e.g., Vbat). This acts to passively equilibrate the charge stored on the capacitance through the patient tissue, as explained further in U.S. Patent Application Publications 2018/0071527 and 2018/0140831. Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs and the electrode nodes ei 39, and between the one or more NDACs and the electrode nodes. Switching matrices allow one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs) within the IPG 10, as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), control circuitry 29, memory, circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. As discussed above, these capacitances 38 will store charge and build up a voltage as current passes through them, thus making charge recovery (active (FIG. 2A) and/or passive (FIG. 2B)) advisable.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with a compatible antenna in the IPG 10 (27a or 27b), such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antennas 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, microcontrollers, FPGAs, DSPs, other digital logic structures, etc., which are capable of executing programs in a computing device. Such control circuitry 92 may contain or be coupled with memory 94 which can store external system software 96 for controlling and communicating with the IPG 10, and for rendering a Graphical User Interface (GUI) 99 (FIG. 5) on a display (61, 71, 88) associated with the external system. In external system 80, the external system software 96 would likely reside in the server 86, while the control circuitry 92 could be present in either or both the server 86 or the terminal 87.

### SUMMARY

A method is disclosed for operating an implantable stimulator device having a plurality of electrodes configured for implantation in a patient's brain. The method may comprise: providing stimulation to the patient as pulses in packets at at least one of the electrodes, wherein the pulses in each packet comprise a number of N pulses issued at a first frequency, wherein N equals two or greater, wherein the packets are issued at a second frequency; receiving a first measurement concerning a first symptom of the patient to assess the effect of the stimulation on the first symptom; receiving a second measurement concerning a second symptom of the patient to assess the effect of the stimulation on the second symptom; in response to the first measurement, adjusting the stimulation by adjusting either or both of (i) the first frequency of the pulses in each packet, or (ii) the number N of pulses provided in each packet; and in response to the second measurement, adjusting the stimulation by adjusting the second frequency at which the packets are issued.

In one example, in response to the first measurement, adjusting both (i) the first frequency of the pulses in each packet, and (ii) the number N of pulses provided in each packet. In one example, the first symptom comprises patient tremor. In one example, the second symptom comprises freezing of gait. In one example, in response to the first measurement, the first frequency is increased. In one example, in response to the first measurement, the number N of pulses provided in each packet is increased. In one example, in response to the second measurement, the second frequency is decreased. In one example, either or both of the first measurement or the second measurement is made using the implantable stimulator device. In one example, either or both of the first measurement or the second measurement is made using at least one sensor separate from the implantable stimulator device. In one example, the at least one sensor comprises a motion sensor. In one example, the at least one sensor is configured to be wearable by the patient. In one example, the first measurement and the second measurement are received at an external system in communication with the implantable stimulator device. In one example, the stimulation is adjusted using telemetry from the external system. In one example, either or both of the first measurement and the second measurement are manually received at a user interface of the external device. In one example, either or both of the first measurement and the second measurement comprise a subjective score indicative of the first symptom. In one example, the stimulation is adjusted automatically. In one example, either or both of the first measurement and the second measurement comprise an objective measurement. In one example, either or both of the first measurement and the second measurement comprise a measurement of motion of the patient. In one example, either or both of the first measurement and the second measurement comprise a neural measurement taken from the patient. In one example, the pulses in each packet are actively-driven monophasic pulses and of a single polarity. In one example, the actively-driven monophasic pulses are followed by passive charge recovery phases. In one example, the pulses in each packet are actively-driven biphasic pulses comprising first and second phases of opposite polarities. In one example, the actively-driven biphasic pulses are followed by passive charge recovery phases. In one example, the pulses in each packet are actively-driven and charge balanced at the at least one of the electrodes. In one example, the implantable stimulator device comprises a case electrode, wherein the stimulation is monopolar using the case electrode as a current return.

A system is disclosed, which may comprise: an implantable stimulator device having a plurality of electrodes configured for implantation in a patient's brain; and control circuitry configured to: provide stimulation to the patient as pulses in packets at at least one of the electrodes, wherein the pulses in each packet comprise a number of N pulses issued at a first frequency, wherein N equals two or greater, wherein the packets are issued at a second frequency; receive a first measurement concerning a first symptom of the patient to assess the effect of the stimulation on the first symptom; receive a second measurement concerning a second symptom of the patient to assess the effect of the stimulation on the second symptom; in response to the first measurement, adjust the stimulation by adjusting either or both of (i) the first frequency of the pulses in each packet, or (ii) the number N of pulses provided in each packet; and in response to the second measurement, adjust the stimulation by adjusting the second frequency at which the packets are issued.

In one example, the control circuitry is in the implantable stimulator device. In one example, the system further comprises an external system in communication with the implantable stimulator device, wherein the control circuitry is in the external system. In one example, the stimulation is adjusted using telemetry from the external system. In one example, either or both of the first measurement and the second measurement are manually received at a user interface of the external system. In one example, either or both of the first measurement and the second measurement comprise a subjective score indicative of the first symptom. In one example, in response to the first measurement, adjust both (i) the first frequency of the pulses in each packet, and (ii) the number N of pulses provided in each packet. In one example, the first symptom comprises patient tremor. In one example, the second symptom comprises freezing of gait. In one example, in response to the first measurement, the first frequency is increased. In one example, in response to the first measurement, the number N of pulses provided in each packet is increased. In one example, in response to the second measurement, the second frequency is decreased. In one example, either or both of the first measurement or the second measurement is made using the implantable stimulator device. In one example, the system further comprises at least one sensor separate from the implantable stimulator device, wherein either or both of the first measurement or the second measurement is made using the at least one sensor. In one example, the at least one sensor comprises a motion sensor. In one example, the at least one sensor is configured to be wearable by the patient. In one example, the stimulation is adjusted automatically. In one example, either or both of the first measurement and the second measurement comprise an objective measurement. In one example, either or both of the first measurement and the second measurement comprise a measurement of motion of the patient. In one example, either or both of the first measurement and the second measurement comprise a neural measurement taken from the patient. In one example, the pulses in each packet are actively-driven monophasic pulses and of a single polarity. In one example, the actively-driven monophasic pulses are followed by passive charge recovery phases. In one example, the pulses in each packet are actively-driven biphasic pulses comprising first and second phases of opposite polarities. In one example, the actively-driven biphasic pulses are followed by passive charge recovery phases. In one example, the pulses in each packet are actively-driven and charge balanced at the at least one of the electrodes. In one example, the implantable stimulator device comprises a case electrode, wherein the stimulation is monopolar using the case electrode as a current return.

A method is disclosed for operating an implantable stimulator device having a plurality of electrodes configured for implantation in a patient's brain. The method may comprise: providing stimulation to the patient as pulses in packets at at least one of the electrodes, wherein the pulses in each packet comprise a number of N anodic pulses issued at a first frequency, wherein N equals two or greater, wherein the packets are issued at a second frequency; receiving at least one measurement concerning at least one symptom of the patient to assess the effect of the stimulation on the at least one symptom; determining that at least one of the symptoms is poor; and in response to determining that the at least one of the symptoms is poor, decreasing an amplitude of the anodic pulses.

In one example, the at least one symptom comprises patient tremor. In one example, the at least one symptom comprises freezing of gait. In one example, in response to determining that the at least one of the symptoms is poor, additionally adjusting the first frequency. In one example, in response to determining that the at least one of the symptoms is poor, additionally adjusting the number N of pulses provided in each packet. In one example, in response to determining that the at least one of the symptoms is poor, additionally adjusting the second frequency. In one example, the at least one measurement is made using the implantable stimulator device. In one example, the at least one measurement is made using at least one sensor separate from the implantable stimulator device. In one example, the at least one sensor comprises a motion sensor. In one example, the at least one sensor is configured to be wearable by the patient. In one example, the at least one measurement is received at an external device in communication with the implantable stimulator device. In one example, the amplitude is decreased using telemetry from the external device. In one example, the at least one measurement is manually received at a user interface of the external device. In one example, at least one measurement comprises a subjective score indicative of the at least one symptom. In one example, in response to determining that the at least one of the symptoms is poor, the amplitude is automatically decreased. In one example, the at least one measurement comprises an objective measurement. In one example, the at least one measurement comprises a measurement of motion of the patient. In one example, the at least one measurement comprises a neural measurement taken from the patient. In one example, the pulses in each packet are actively-driven monophasic pulses. In one example, the actively-driven monophasic pulses are followed by passive charge recovery phases. In one example, the implantable stimulator device comprises a case electrode, wherein the stimulation is monopolar using the case electrode as a current return.

A system is disclosed, which may comprise: an implantable stimulator device having a plurality of electrodes configured for implantation in a patient's brain; and control circuitry configured to: provide stimulation to the patient as pulses in packets at at least one of the electrodes, wherein the pulses in each packet comprise a number of N anodic pulses issued at a first frequency, wherein N equals two or greater, wherein the packets are issued at a second frequency; receive at least one measurement concerning at least one symptom of the patient to assess the effect of the stimulation on the at least one symptom; determine that at least one of the symptoms is poor; and in response to determining that the at least one of the symptoms is poor, decrease an amplitude of the anodic pulses.

In one example, the control circuitry is in the implantable stimulator device. In one example, the system further comprises an external system in communication with the implantable stimulator device, wherein the control circuitry is in the external system. In one example, the amplitude is decreased using telemetry from the external device. In one example, the at least one measurement is manually received at a user interface of the external device. In one example, the at least one measurement comprises a subjective score indicative of the at least one symptom. In one example, the at least one symptom comprises patient tremor. In one example, the at least one symptom comprises freezing of gait. In one example, in response to determining that the at least one of the symptoms is poor, additionally adjust the first frequency. In one example, in response to determining that the at least one of the symptoms is poor, additionally adjust the number N of pulses provided in each packet. In one example, in response to determining that the at least one of the symptoms is poor, additionally adjust the second frequency. In one example, the at least one measurement is made using the implantable stimulator device. In one example, the system further comprises at least one sensor separate from the implantable stimulator device, wherein the at least one measurement is made using the at least one sensor. In one example the at least one sensor comprises a motion sensor. In one example, the at least one sensor is configured to be wearable by the patient. In one example, in response to determining that the at least one of the symptoms is poor, the amplitude is automatically decreased. In one example, the at least one measurement comprises an objective measurement. In one example, the at least one measurement comprises a measurement of motion of the patient. In one example, the at least one measurement comprises a neural measurement taken from the patient. In one example, the pulses in each packet are actively-driven monophasic pulses. In one example, the actively-driven monophasic pulses are followed by passive charge recovery phases. In one example, the implantable stimulator device comprises a case electrode, wherein the stimulation is monopolar using the case electrode as a current return.

### BRIEF DESCRIPTION OF THE DRAWINGS

FIG. 1 shows an Implantable Pulse Generator (IPG), in accordance with the prior art.

FIGS. 2A and 2B show example of stimulation pulses (waveforms) producible by the IPG using actively-driven biphasic and monophasic pulses respectively.

FIG. 3 shows an example of stimulation circuitry useable in the IPG, in accordance with the prior art.

FIG. 4 shows various external systems capable of communicating with and programming stimulation in an IPG, in accordance with the prior art.

FIGS. 5A-8 show various N-let waveforms having high and low frequency components that can be used for DSB therapy to treat different DBS symptoms.

FIG. 9 shows a graphical user interface of an external system that can be used to program an IPG with any of the waveforms of FIGS. 5A-8.

FIG. 10 shows an algorithm that can be used to adjust N-let waveforms used to provide DBS therapy.

FIG. 11 shows portions of the algorithm that can be used to adjust other stimulation parameters, like amplitude.

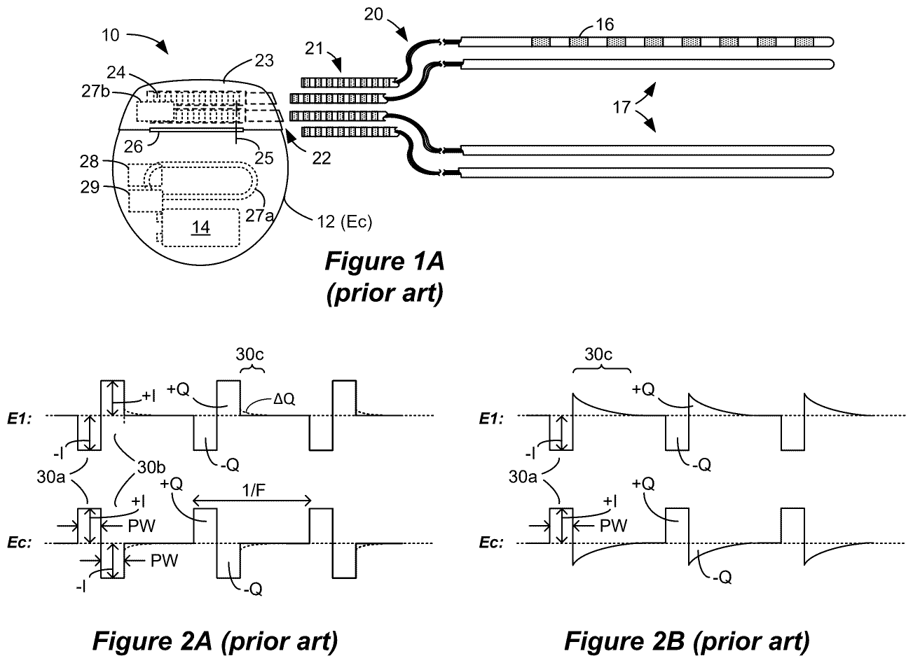

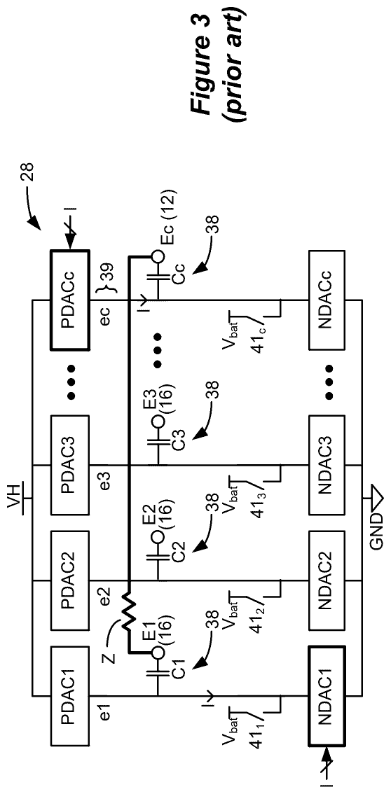

### DETAILED DESCRIPTION

DBS patients typically suffer from a host of symptoms. In particular, such patients can suffer from tremors. Evidence and experience suggest that tremors can be most effectively treated by using higher frequency pulses, such as 200 Hz or greater. Another symptom affecting such patients is known as freezing of gait, which is a phenomenon in which a patient seems to get stuck or frozen while walking. Evidence and experience suggest that freezing of gait can be treated by pulses with a lower frequency, such as 100 Hz or less.

It can then be difficult to tailor neuromodulation therapies for such DBS patients, because pulses increasing or decreasing the frequency can positively affect one symptom while negatively affecting another. The inventors address this issue by providing neurostimulation for DBS patients having both high and low frequency components. The high and low frequency components are independently adjustable, thus allowing these frequencies to be tailored to best treat a patient's symptoms.

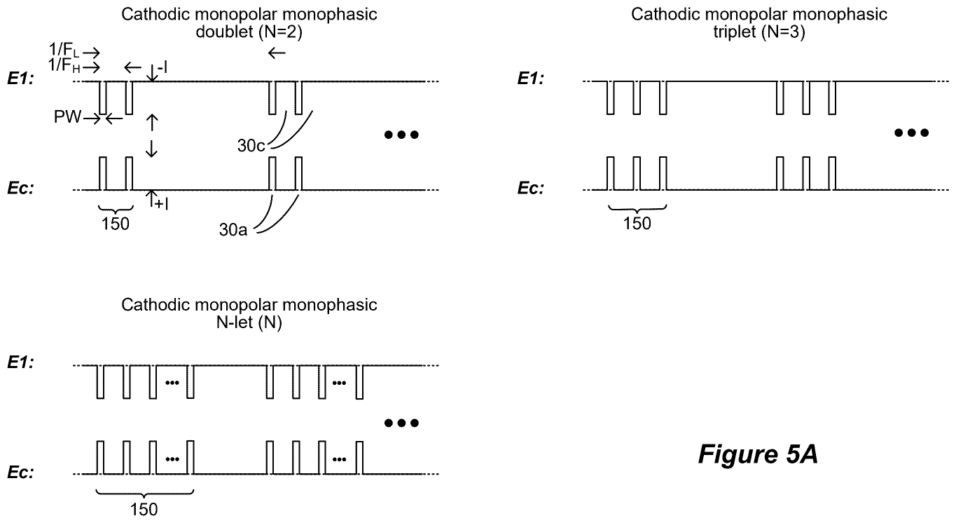

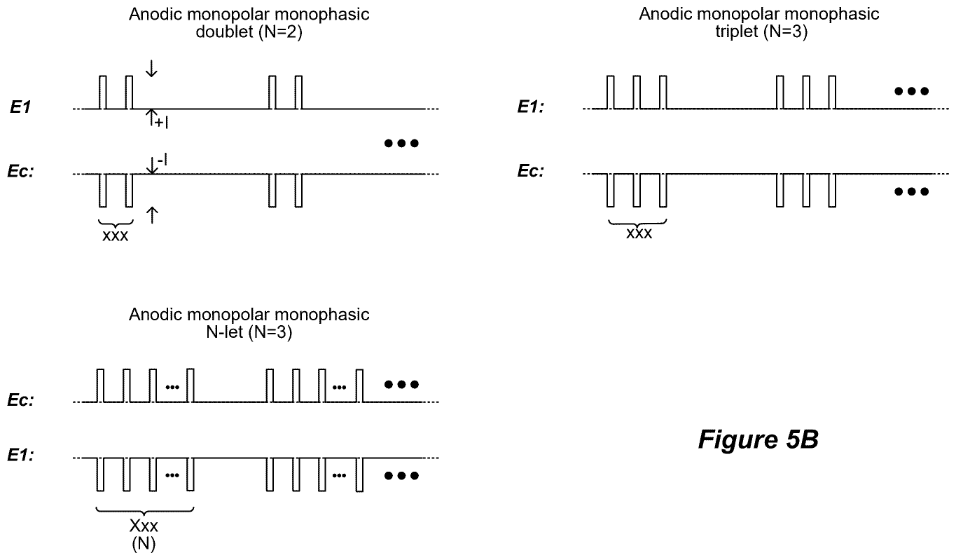

FIG. 5A shows a first example of waveforms that can be provided to a DBS patient, and having both high- and low-frequency components. In this, and other examples, pulses are provided in packets 150, which may be referred to as “N-lets” in accordance with the number of pulses in the packet. FIG. 5A shows examples in which the packets 150 include 2 pulses (a doublet), 3 pulses (a triplet), and some number N of pulses (N-let). The pulses in each packet 150 are issued at a high frequency, F_{H}. The packets 150 themselves are issued at a low frequency, F_{L}. These frequencies F_{H} and F_{L} are tailorable to treat particular symptoms of the patient, as discussed further below.

In the example of FIG. 5A, the pulses in each packet are monophasic, similar to what was shown earlier with respect to FIG. 2B, and have only one actively-driven phase 30a. These pulses (i.e., phase 30a) have a constant current amplitude I and a pulse width PW. Further, the pulses shown in FIG. 5A are monopolar, which as discussed earlier, means that the electrode array 17 includes a single pole. In this case, this single pole is formed using a single electrode (E1) in the electrode array, but as noted earlier, a plurality of electrodes can be driven with the same polarity to form a single pole. In the example shown in FIG. 5A, the pulses provided (in the electrode array 17) are cathodic with amplitude −I), with the case electrode Ec being actively driven with pulses of opposite polarity (anodic, +I) to provide a current return. However, anodic pulses could also be used as shown in the examples of FIG. 5B. The use of anodic pulses in a DBS context has been shown to have certain benefits over the use of cathodic pulses, although higher amplitudes +I may be required. See, e.g., U.S. Patent Application Publications 2019/0329039; 2019/0329024; A. D. Kirsch et al., “Anodic Versus Cathodic Neurostimulation of the Subthalamic Nucleus: A Randomized-Controlled Study of Acute Clinical Effects,” Parkinsonism and Related Disorders, 55, pp. 61-67 (2018).

Because the pulses in FIGS. 5A and 5B are monophasic, charge recovery may be an issue, because the actively-driven phase 30a is not followed by an actively-driven charge recovery phase (e.g., 30b, FIG. 2A). As such, it may be useful to provide passive charge recovery. As explained earlier, passive charge recovery preferably occurs after a given pulse (30a) during durations 30c. Passive charge recovery may also occur after each packet 150, i.e., during the duration between successive packets.

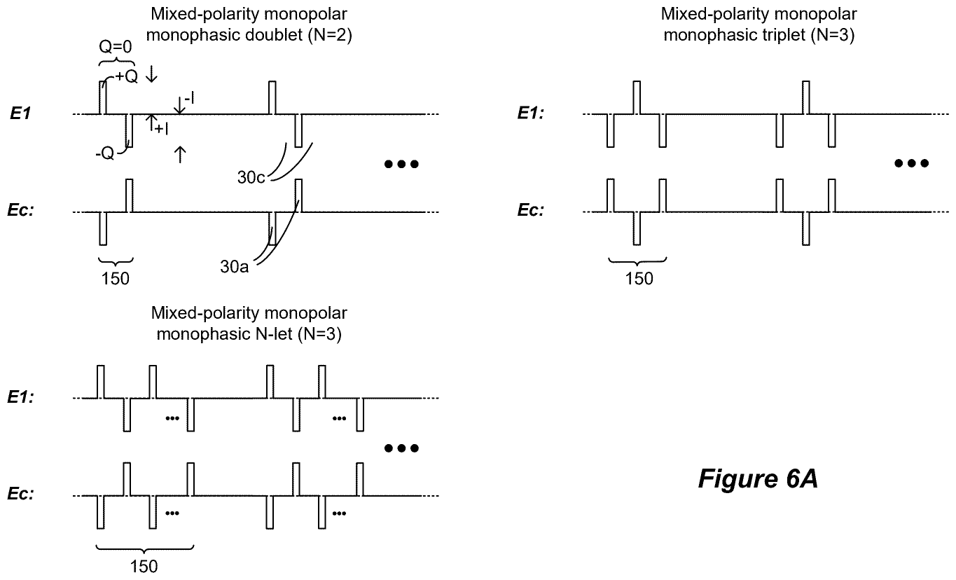

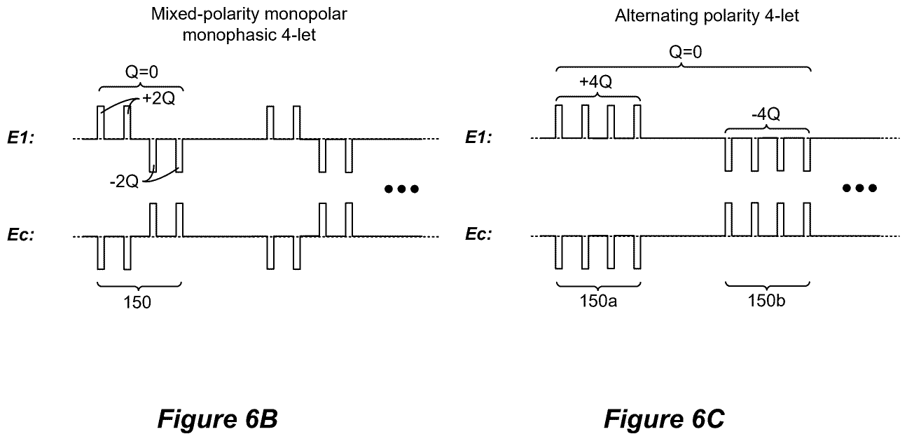

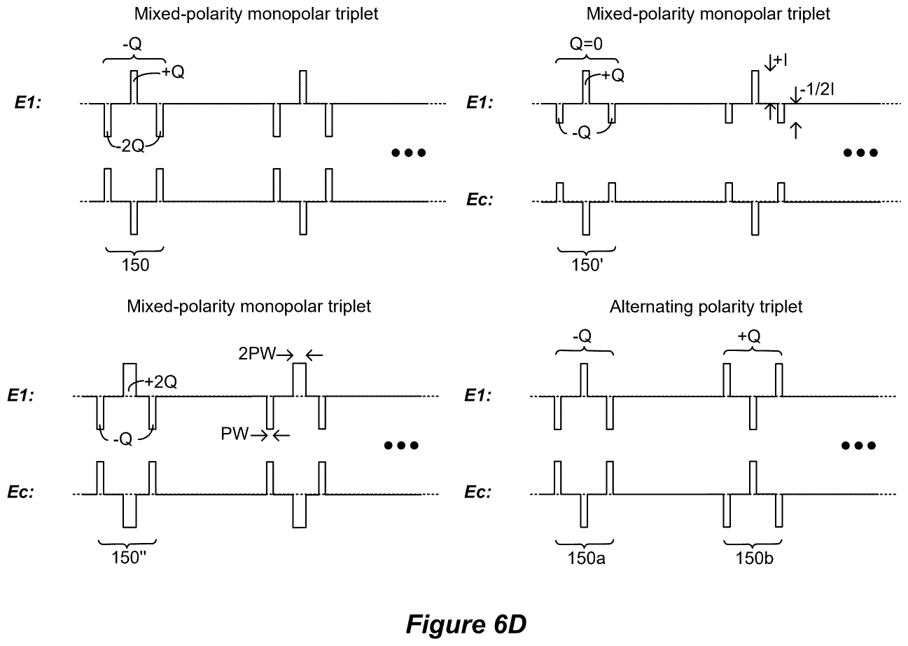

The polarity of the pulses within a packet 150 may also be varied. For example, as shown in FIG. 6A, doublet, triplet, and N-let packets 150 are shown having at least one monophasic anodic pulse and at least one monophasic cathodic pulse that are alternated at the high frequency F_{H}. Using pulses with different polarities can be useful both therapeutically, and because opposite polarity pulses can actively recover injected charge. For example, in the doublet example of FIG. 6A, the first anodic pulse injects a charge of +Q (+I*PW), with the following cathodic pulse injecting an equal by opposite charge of −Q(−I*PW). As such, the net charge injected at each electrode (E1, Ec) during each packet 150 is Q=0. Because charge is recovered in this example, the use of passive charge recovery (30c) may not be necessary, although it could also be used (e.g., following each pulse, or following each packet 150) as shown in FIG. 6A. FIG. 6B shows another example in which the polarity of the pulses in each packet 150 are varied, but not necessary in an alternating fashion. In this example, two anodic pulses (+2Q) are followed by two cathodic pulses (+2Q) at high frequency F_{H}. Notice that this also beneficially results in a charge balanced packet 150. Again, passive charge recovery (30c) may not be necessary in this instance, but could still be used.

Packets with different pulses can also be used to provide charge balance at each electrode. For example, in FIG. 6C, four monophasic anodic pulses (at F_{H}) are provided (+4Q) in a first packet 150a, and four monophasic cathodic pulses (F_{H}) are provided (−4Q) in a second packet 150a. In this example, the packets 150a and 150b are interleaved in time at a frequency of F_{L}. While neither packet 150a nor 150b is itself charge balanced, both packets considered together provide charge balance at the electrodes. Passive charge recovery (30c) may not be necessary in this instance, but could still be used.

If necessary, pulses provided in a packet 150 can be modified to charge balance the packet. Consider for example, the triplet shown at the upper left in FIG. 6D. This triplet includes a monophasic cathodic, anodic, and cathodic pulses in order, such that the packet 150 has a net charge of −2Q++Q=−Q. If such charge imbalance is undesirable, the pulses in the packet 150 can be modified while still issuing at high frequency F_{H}. This can occur by modifying either the amplitude or pulse width of some of the pulses in the packet. For example, in packet 150′ (upper right), the amplitude of the cathodic pulses is halved (−1/2I), which sets the net charge of the pulses in packet 150′ to zero. As well as being charge balanced, this can be therapeutically useful, because as discussed above, larger-amplitude anodic pulses have been shown to be useful in the DBS context. In packet 150″ (lower left), the pulse width of the anodic pulse is doubled, which again sets the net charge of the pulses in packet 150″ to zero. The lower right shows an example in which different alternating packets 150a and 150b are used to promote charge balance, in which the polarity of packet 150a is inverted compared to packet 150b. Similarly to the example of FIG. 6C, while neither packet 150a nor 150b is itself charge balanced, both packets considered together provide charge balance at the electrodes.

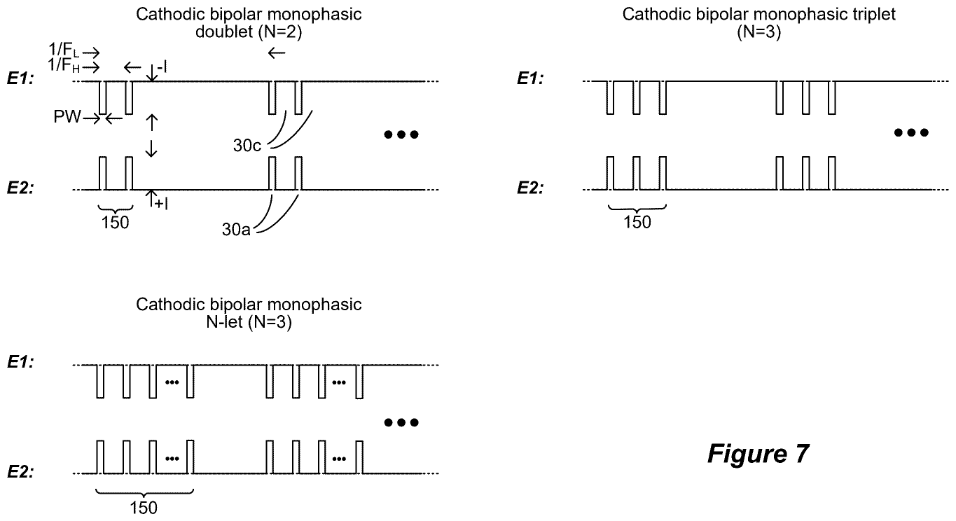

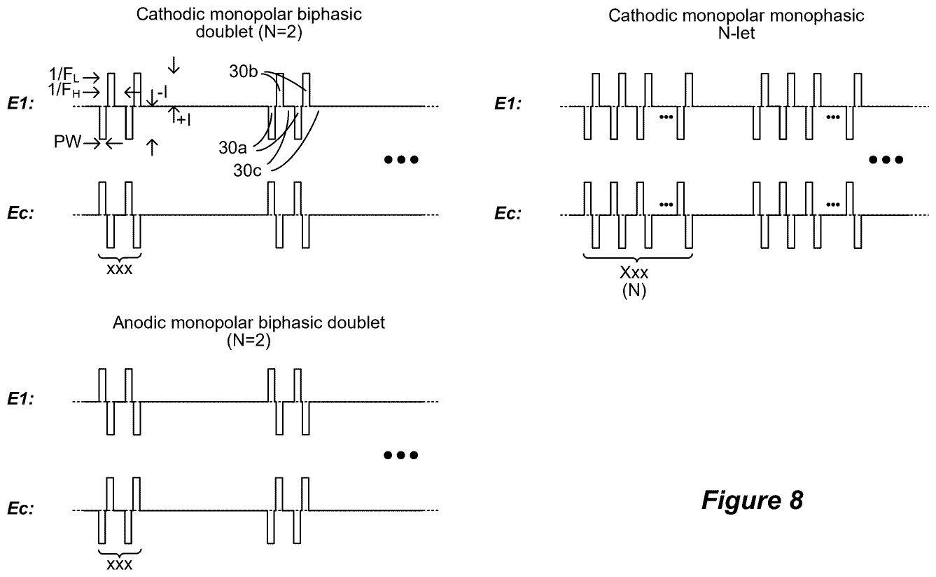

FIGS. 7 and 8 show other types of N-let pulses that can be used in a DBS setting having the high and low frequencies F_{H} and F_{L} described earlier. In FIG. 7, bipolar stimulation is used, which as noted earlier involve forming a plurality of poles in the electrode array 17. In the example shown, monophasic cathodic pulses are provided in packets 150 to form a cathode pole at electrode E1, while monophasic anodic pulses are used to form an anode pole at electrode E2. This is just a simple example, and as noted earlier, more than one electrode can be active in the electrode array 17 to form each pole. Stimulation could also be multipolar in a DBS context. As in some earlier examples, the packets 150 shown in FIG. 7 include a doublet, triplet, and a generic N-let of pulses. As in earlier examples, passive charge recovery 30c can be used after the pulses and packets. The polarity of the pulses in FIG. 7 could also be modified, or mixed, in each packet 150, as described previously.

FIG. 8 shows another modification in which biphasic pulses are used in the packets 150. Monopolar stimulation is shown in which a single pole is formed in the electrode array 17 (at E1), using the case electrodes Ec as a return, but bipolar (FIG. 7) or multipolar stimulation could be used as well. As discussed earlier, biphasic pulses involve actively driving a current of opposite polarities in two different phases 30a and 30b (see FIG. 2A) of each pulse. Passive charge recovery can also be used (30c), but this may be unnecessary due to the active charge recovery that phase 30b provides. Because biphasic pulses can be inherently charge balanced between their phases 30a and 30b, the packets 150 can also be inherently charge balanced (net charge=0) at each of the electrodes. As in other example, the biphasic pulses in FIG. 8 are issued in each packet 150 at high frequency F_{H}, while the packets 150 themselves are issued at low frequency F_{L}.

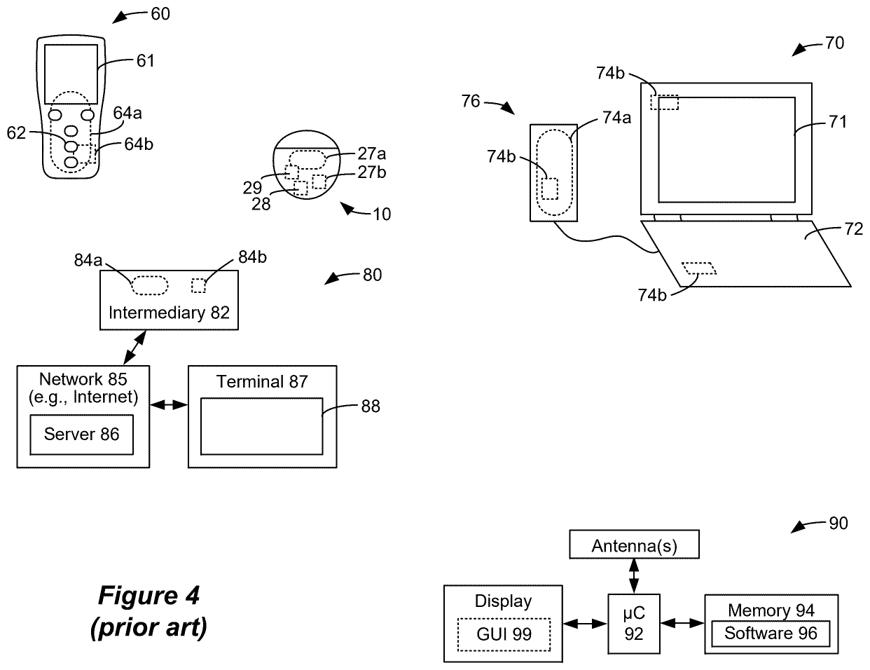

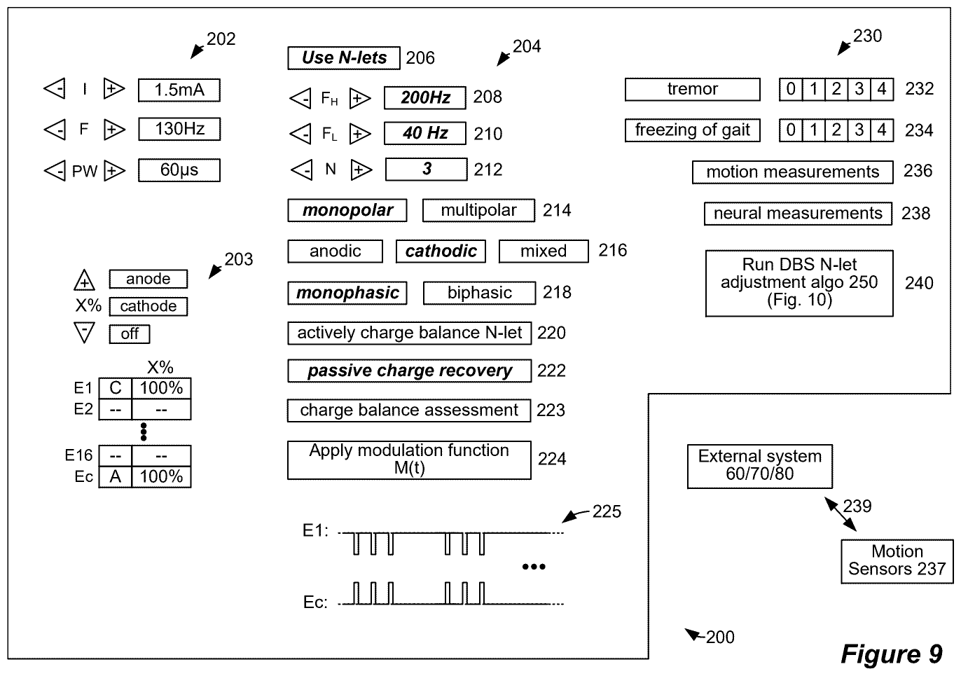

FIG. 9 shows a simple example of GUI 200 useable in an external system to program the use of N-lets of pulses for a DBS patient. GUI 200 is an example of GUI 99 that can be used to program stimulation for the patient, as discussed earlier. Like GUI 99, GUI 200 can comprise at least a part of external system software 96 executable in or on an external system such as 60, 70, or 80 described earlier (FIG. 4). Such software 96 can comprise instructions in a non-transitory computer readable medium such as a solid state, optical, or magnetic memory, such as memory 94 associated with or downloadable to the external system.

GUI 200 includes a tonic waveform interface 202 that allows the shape and timing of tonic stimulation pulses such as those shown in FIGS. 2A and 2B to be selected or adjusted. For example, waveform interface 104 allows a user to select an amplitude (e.g., a current I), a frequency (F), and a pulse width (PW) of tonic stimulation pulses. GUI 200 may also include an electrode configuration interface 203 which allows the user to select a particular electrode configuration specifying which electrodes should be active to provide the stimulation, and with which polarities and relative magnitudes. In this example, the electrode configuration interface 203 allows the user to select whether an electrode should comprise an anode (A) or cathode (C) or be off, and allows the amount of the total anodic or cathodic current +I or −I (specified in the waveform interface 202) that each selected electrode will receive to be specified in terms of a percentage, X. For example, in FIG. 9, Electrode E1 is specified to be a cathode (C) that receives X=100% of the current I as a cathodic current −I during a first actively driven phase (e.g., 30a). The case electrode 12 Ec is specified to be an anode (A) that receives X=100% of the current I as an anodic current +I (again, during phase 30a), which as noted earlier provides an example of monopolar stimulation. Although not shown, during monopolar stimulation, the cathodic current −I could be split between different electrodes forming a single cathode pole. For example, electrodes E1 and E2 could both be selected as cathodes, each splitting the cathodic current −I differently (e.g., X=60% and 40%).

Of more relevance to this application, GUI 200 also includes an N-let waveform interface 204 allowing DBS stimulation to be defined in N-lets, as disclosed in the examples shown earlier (FIGS. 5A-8). Option 206 allows the user to select the use of N-lets, and when selected may also allow for the selection of other options that follow. Option 208 allows the high frequency F_{H} of the pulses in each packet 150 to be set, while option 210 allows the low frequency F_{L} of the packets themselves to be set. Option 212 allows the number of pulses in each packet 150 (N) to be set. Note that when N-let stimulation is selected (206), the tonic frequency F in tonic waveform interface 202 may become moot, and hence the option to adjust this tonic frequency may be removed or deactivated (or replaced with one of F_{L} or F_{H}). As discussed subsequently, parameters F_{H}, F_{L}, and N may also be automatically adjusted in accordance with DBS N-let adjustment algorithm 250.

Option 214 allows the user to select the use of monopolar (e.g., FIGS. 5A-6D, 8) or multipolar (e.g., FIG. 7) stimulation. Option 216 allows the polarity of the pulses in the N-lets to be set, either as anodic (FIG. 5B), cathodic (FIGS. 5A, 7), or mixed (FIG. 6A-6D). Note that these options 214 and 216 may control or be controlled by electrode configuration interface 203. For example, if electrode E1 is set as a cathode and case electrode Ec is set as an anode in interface 203, then “monopolar” and “cathodic” may be auto-populated as selected options in N-let waveform interface 204. Likewise, selecting cathodic and monopolar in interface 204 could for example cause the case electrode Ec to be auto populated as a return anode (X=100%).

Option 218 allows monophasic (e.g., FIG. 5A-7) or biphasic (FIG. 8) stimulation pulses to be selected for use in the defined N-lets. Option 220 allows the defined pulses in the N-let to be charge balanced at the electrodes. Such charge balancing can be affected in different manners, such as discussed earlier with respect to FIG. 6D. Option 222 allows passive charge recovery (phases 30c) to be used after the pulses in the N-lets. Option 223 allows the user to select a charge balance assessment, where the system analyzes and estimates whether a passive pulse (during phases 30c) will cause more or less excitation than the active pulses. If a passive pulse would inadvertently cause a higher excitation (which is generally not desired), the GUI 200 could display an indication to notify the user, and/or propose an adjustment to the stimulation to reduce the likelihood of an unexpected excessively-intense passive pulse. Option 223 may rely on the use of measured electrode impedances. Lastly, visualization interface 225 can provide an example of what the waveform defined using N-let waveform interface 204 will look like as a function of time. Note that other waveform parameters depicted in the visualization interface 225, such as amplitude I and pulse width PW, may be specified in the tonic waveform interface 202.

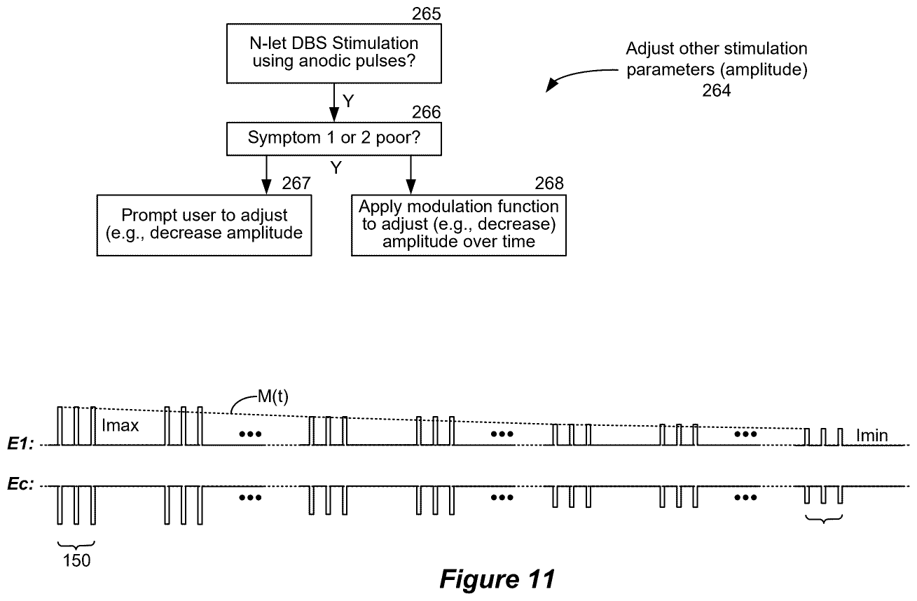

The N-let waveform interface 204 could include further inputs relevant to defining N-lets of pulses as useful for DBS therapy. For example, it may be beneficial to adjust (e.g., reduce) the amplitude the pulses as a function of time, and as such, an option 224 can be included to modulate the amplitude of the pulses. As described further below with reference to FIG. 11, option 224 may apply a modulation function (M(t)) that alters the amplitude of the pulses over time. Although not shown, option 224 could include other inputs as well. For example, option 224 could allow different modulation functions (M1(t), M2(t), etc.) to be chosen. Option 224 can also include inputs to specify a maximum and/or minimum amplitude, such as a maximum or minimum amplitude that the modulation function M(t) will impart to the pulses. Option 224 can also more generically be used to define the modulation function. Manners in which a modulation function can be applied to affect the amplitude of pulses is disclosed in U.S. Patent Application Publication 2022/0347479, which is incorporated herein by reference.

GUI 200 preferably also includes a feedback interface 230 which allows the effectiveness of N-let DBS therapy to be measured, and further, for N-let therapy to be adjusted in a closed loop fashion based on such measurements. The feedback interface 230 includes different means for receiving measurements indicative of the effectiveness of DBS therapy, and such measurements may be determined subjectively or objectively. An example of subjective measurements includes entry of scores obtained from applying Unified Parkinson's Disease Rating Scales (UPDRSs). As is known, such scales allow a patient or clinician to subjectively numerically score a given symptom from 0 (best) to 4 (worst) based on patient observation or feedback. In the example of FIG. 9, for simplicity, only two such scales are shown for assessing the symptoms of tremor and freezing of gait, which are respectively input at options 232 and 234 from time to time based on patient observation. However, scores for other symptoms could be input as well.

Options 236 and 238 allow for the objective measurement of symptoms such as tremor and gait. Option 236 allows motion measurements to be made objectively using one or more motion sensors 237. Such sensors 237 can be externally worn by the patient to measure a particular symptom. For example, a motion sensor 237 used to measure tremor may be wearable on the patient's hand or wrist. A motion sensor 237 to measure freezing of gait may be wearable on the patient's leg or foot. Motion sensors 237 are preferably able to wirelessly communicate their measurements to the external system in which GUI 200 is running via a wireless link 239 as shown. A motion sensor 237 may also be included with, or in communication with, the patient's IPG 10.

Option 238 allows neural measurements to be taken. Such neural measurements can comprise measurements taken by the IPG 10 itself, for examples signals measured at the IPG 10's electrodes, or measurements taken by a system independent from the IPG 10 (e.g., EEG equipment). Such neural measurements can comprise neural responses to provided stimulation (such as evoked potentials or responses), or background neural measurements taken in the absence of simulation (such as local field potentials (LFPs)). Like option 236, neural measurements at option 238 may be wirelessly communicated to the external system executing GUI 200, for example, from the antenna (27a and/or 27b, FIG. 4) in the IPG 10.

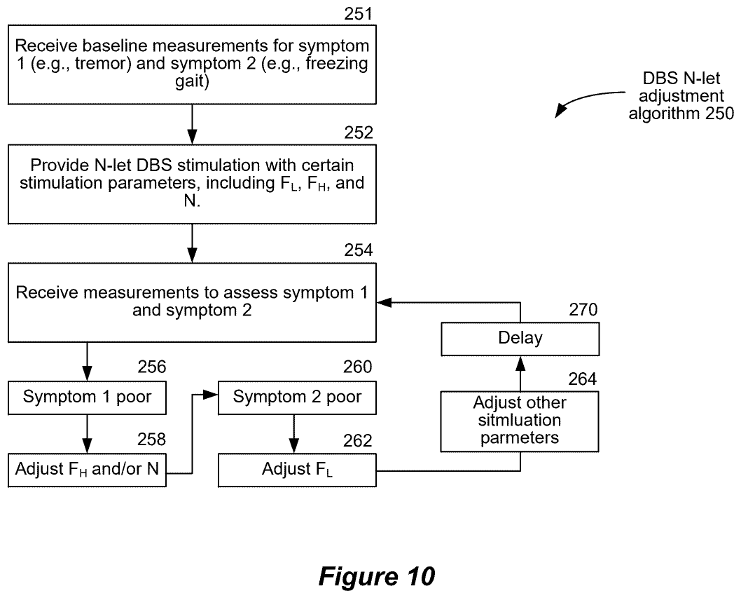

Option 240 allows the user to execute a DBS N-let algorithm 250 to adjust N-let stimulation in accordance with the subjective or objective measurements indicating symptom severity. This algorithm 250 is shown in further detail in FIG. 10. Note that algorithm 250 can operate exclusively in the external system associated with GUI 200 (in association with its control circuitry 92), exclusively in the IPG 10 (in association with its control circuitry 29), or can operate in part in both the external system and the IPG 10. Manners in which algorithm 250 can be modified depending on where it is executed are discussed further below.

Algorithm 250 preferably starts at step 251 by receiving measurement of various patient symptoms. As noted earlier, many symptoms can be measured at this step, but for simplicity algorithm 250 focuses on two symptoms (symptoms 1 and 2), which may respectively comprise tremor and freezing of gait. Step 251 is optional, but can be useful in the algorithm 250 to establish baseline measurement for these symptoms before N-let DBS therapy is provided, and to allow the algorithm 250 to gauge how these measurements are affected once N-let DBS therapy has begun. As noted earlier, the measurements at step 251 can be subjective (e.g., UPDRS scores) and/or objective (e.g., motion or neural measurements). If not automatically received at the external system, these measurements can be entered manually using options 232-238 in the GUI 200. If algorithm 250 operates in the IPG 10, these measurements can be telemetered to the IPG 10 (e.g., from the external system or sensor(s) 237) as necessary (i.e., if not already taken by the IPG 10).

Next, N-let DBS therapy is provided by the IPG 10 with certain stimulation parameters (252). This can comprise any of the parameters described earlier in conjunction with GUI 200, including amplitude I, pulse width PW (202), the active electrodes (203), and the various options described in N-let waveform interface 204. Specifically, the parameters include the low frequency F_{L} at which the packets 150 are issued, the high frequency F_{H} at which the pulses in each packet 150 are issued, and the number N of pulses in each of the packets. The external system in which GUI 200 is running can telemeter the stimulation parameters to the IPG 10, as discussed earlier.

Once stimulation has begun at the IPG 10, measurements to assess symptoms 1 and 2 can be received by the algorithm 250 (254). This can occur as described earlier (e.g., 251), either automatically or using options 232-238 in the GUI 200. If algorithm 250 operates in the IPG 10, these measurements can be telemetered to the IPG 10 (e.g., from the external system or sensor(s) 237).

Once relevant measurements are received by the algorithm 250, they can be evaluated to assess symptoms 1 and 2, and possibly to adjust the N-let DBS therapy. For example, measurements relevant to symptom 1 can be assessed first to see if they indicate that symptom 1 is poor for the patient (256). Whether symptom 1 is poor for the patient can be determined in different ways. For example, the measurement may simply indicate a poor absolute measurement for symptom 1 (e.g., a high tremor), such as a measurement that exceeds a pre-set threshold. Baseline measurements taken earlier (251) can also be considered, with a poor measurement for symptom 1 being determined as one that is worse than the baseline established for that symptom, or a measurement for that symptom that isn't improved significantly using the stimulation (252). A poor measurement for symptom 1 can also be determined based on previous measurements for that symptom taken as the algorithm 250 iterates. Such historical measurements may indicate that symptom 1 is worsening, or not improving significantly with stimulation.

If symptom 1 is poor, adjustments can be made to high-frequency aspects of the N-let DBS therapy (258). For example, if symptom 1 comprises tremor, poor tremor measurements (e.g., from one or more of motion sensors 237) may warrant adjusting F_{H}. In particular, it may be useful to increase F_{H}. As noted earlier, evidence and experience suggest that tremors can be most effectively treated by using higher frequency pulses, such as 200 Hz or greater, suggesting that the use of such frequencies in each packet 150 will better treat tremor symptoms. F_{H} could however also be decreased at this step. Alternatively, or additionally, poor tremor measurements may warrant adjusting the number of pulses N that are issued in each packet 150 at this high frequency F_{H}, and in particular it may be useful to increase N. Even though the high frequency pulses in each packet 150 are not free running, increasing N will establish this higher frequency treatment for longer periods of time, which may further assist in treating (e.g.) tremor. N could however also be decreased at this step. The decision to adjust F_{H} and/or N can occur in the external system or the IPG 10, depending where the algorithm 250 is operating. If algorithm 250 is operating in the external system, new values for F_{H} and/or N, or new data necessary to cause the IPG 10 to provide waveform consistent with those parameters, can be telemetered from the external system to the IPG 10. Adjustment of F_{H} and/or N can be made automatically by the algorithm 250, or the user can be prompted in GUI 200 to manually change these parameters at options 208 and 212 (FIG. 9). In this regard, the algorithm 250 may suggest values for these parameters which the user can adopt in the GUI 200.

Another symptom (symptom 2) can be similarly evaluated by the algorithm 200, to possibly adjust low frequency aspects of the N-let DBS therapy. For example, measurements relevant to symptom 2 can be assessed first to see if they indicate that symptom 2 is poor for the patient (260). Again, this can be determined in different ways, as explained earlier at step 256. If symptom 2 is poor, adjustment to the stimulation can be made to low-frequency aspects of the N-let DBS therapy (262). For example, if symptom 2 comprises freezing gait, poor freezing gait measurements (e.g., from one or more of motion sensors 237) may warrant adjusting F_{L}. In particular, it may be useful to decrease F_{L}. As noted earlier, evidence and experience suggest that freezing gait can be treated by pulses with a lower frequency, such as 100 Hz or less. This suggests that issuing each packet 150 at such low frequencies will better treat freezing of gait symptoms, even though the pulses issued in packet 150 are not singular tonic pulses. F_{L} could however also be decreased at this step. As discussed earlier, the decision to adjust F_{L} can occur in the external system or the IPG 10, depending where the algorithm 250 is operating. If algorithm 250 is operating in the external system, a new value for F_{L}, or new data necessary to cause the IPG 10 to provide a stimulation waveform consistent with that parameter, can be telemetered from the external system to the IPG 10. Adjustment of F_{L} can be made automatically by the algorithm 250, or the user can be prompted in GUI 200 to manually change this parameter at option 210 (FIG. 9). In this regard, the algorithm 250 may suggest a value for this parameter which the user can adopt in the GUI 200.

In an optional step 264, the algorithm 250 can adjust other stimulation parameters beyond parameters F_{L}, F_{H}, and N that were made adjustable earlier. Such parameters may include those originally set for N-let DBS stimulation at step 252 (e.g., polarity (which phases are anodic and which are cathodic), amplitude I and pulse width PW). As with adjustments to F_{L}, F_{H}, and N, adjustment to other stimulation parameters at step 264 may be automatic or manual via the GUI 200.

FIG. 11 shows one manner in which other stimulation parameters can be adjusted at step 264. In this example, the amplitude of anodic pulses are adjusted. This is particularly useful, because evidence suggests that better control of patient symptoms may be achieved if the amplitude of anodic stimulation is reduced over time. Thus in step 265, the algorithm 250 inquires whether anodic pulses are being used for N-let DBS stimulation. If so, symptoms—e.g., either or both of symptoms 1 and 2—are assessed using the measurements described earlier, and if these symptoms are poor, the amplitude of the stimulation is adjusted. Such stimulation adjustment can occur in different manners, as shown in steps 267 and 268. In step 267, the user is prompted by the GUI 200 to consider adjusting the amplitude, and further may be specifically prompted to reduce the amplitude. In response, the user may manually adjust the amplitude I, such as by using tonic waveform interface 202. Additionally, or alternatively, in step 268 the amplitude can be automatically adjusted in accordance with a modulation function, M(t). As noted earlier, this modulation function may be entered using option 224 in the interface 204. The bottom of FIG. 11 shows the effect of modulating the amplitude of anodic pulses using a modulation function, and shows the amplitude decreasing over time from a maximum amplitude (Imax) to a minimum amplitude (Imin). As noted earlier, these maximum and minimum amplitude can also be entered at option 224.

Adjustment of other stimulation parameters at step 264 can occur in different manners depending on the types of pulses used. For example, different adjustment may be provided to cathodic pulses, such as the use of a different modulation function. The parameter of pulses width (PW) could be modified as well. Step 264 may also allow the polarity of the pulses to be varied or modified in other examples.

At step 270, the algorithm 250 may delay before receiving further measurements relevant to symptoms 1 and 2 (254). Such a delay keeps the algorithm 250 from receiving measurements, and potentially adjusting the DBS N-let stimulation, unnecessarily quickly. The delay instituted at step 270 may comprise about a minute in one example. Delay 270 may be constant, particularly if measurements at step 254 are taken automatically at regular intervals. Delay 270 may also be variable, and may simply comprise a time between subsequent measurements at step 254, such as when the user decides to score or update the score of, certain symptoms (FIG. 8, options 232, 234).

While the algorithm 250 in FIG. 10 shows the adjustment of N-let stimulation based on assessing two different symptoms (256, 260), stimulation could also be adjusted based on assessment of just a single symptom. For example, steps 260 and 262 could be omitted, allowing only symptom 1 to be assessed (256), resulting in adjustments to high frequency aspects of the stimulation (258, F_{H} and N). Similarly, steps 256 and 258 could be omitted, allowing only symptom 2 to be assessed (260), resulting in adjustments to low frequency aspects of the stimulation (F_{L}). Step 256 to 262 could also be omitted entirely, allowing other stimulation parameters to be adjusted at step 264. Although not shown, different iterations of the algorithm could allow for different adjustments at different times. For example, a first iteration could allow adjustments to only high frequency aspects (steps 256, 258). A second iteration could allow adjustment to only low frequency aspects (steps 260, 262). A third iteration could allow adjustment to only other stimulation parameters (step 264). In other words, the algorithm 250 may allow adjustment to different, specific stimulation parameters at different times, rather than potentially allowing all parameters to be adjusted.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for operating an implantable stimulator device having a plurality of electrodes configured for implantation in a patient's brain, comprising:
providing stimulation to the patient as pulses in packets at at least one of the electrodes, wherein the pulses in each packet comprise a number of N pulses issued at a first frequency, wherein N equals two or greater, wherein the packets are issued at a second frequency;
receiving a first measurement concerning a first symptom of the patient to assess the effect of the stimulation on the first symptom;
receiving a second measurement concerning a second symptom of the patient to assess the effect of the stimulation on the second symptom;
in response to the first measurement, adjusting the stimulation by adjusting either or both of (i) the first frequency of the pulses in each packet, or (ii) the number N of pulses provided in each packet; and
in response to the second measurement, adjusting the stimulation by adjusting the second frequency at which the packets are issued.

2. The method of claim 1, wherein in response to the first measurement, adjusting both (i) the first frequency of the pulses in each packet, and (ii) the number N of pulses provided in each packet.

3. The method of claim 1, wherein the first symptom comprises patient tremor.

4. The method of claim 3, wherein the second symptom comprises freezing of gait.

5. The method of claim 1, wherein in response to the first measurement, the first frequency is increased.

6. The method of claim 1, wherein in response to the first measurement, the number N of pulses provided in each packet is increased.

7. The method of claim 1, wherein in response to the second measurement, the second frequency is decreased.

8. The method of claim 1, wherein either or both of the first measurement or the second measurement is made using the implantable stimulator device.

9. The method of claim 1, wherein either or both of the first measurement or the second measurement is made using at least one sensor separate from the implantable stimulator device.

10. The method of claim 9, wherein the at least one sensor comprises a motion sensor, or wherein the at least one sensor is configured to be wearable by the patient.

11. The method of claim 1, wherein the first measurement and the second measurement are received at an external system in communication with the implantable stimulator device.

12. The method of claim 11, wherein the stimulation is adjusted using telemetry from the external system.

13. The method of claim 11, wherein either or both of the first measurement and the second measurement are manually received at a user interface of the external device, wherein either or both of the first measurement and the second measurement comprise a subjective score indicative of the first symptom.

14. The method of claim 1, wherein the stimulation is adjusted automatically.

15. The method of claim 1, wherein either or both of the first measurement and the second measurement comprise an objective measurement.

16. The method of claim 1, wherein the pulses in each packet are actively-driven monophasic pulses and of a single polarity.

17. The method of claim 1, wherein the pulses in each packet are actively-driven biphasic pulses comprising first and second phases of opposite polarities.

18. The method of claim 1, wherein the pulses in each packet are actively-driven and charge balanced at the at least one of the electrodes.

19. The method of claim 1, wherein the implantable stimulator device comprises a case electrode, wherein the stimulation is monopolar using the case electrode as a current return.

20. A system, comprising:
an implantable stimulator device having a plurality of electrodes configured for implantation in a patient's brain; and
control circuitry configured to:
provide stimulation to the patient as pulses in packets at at least one of the electrodes, wherein the pulses in each packet comprise a number of N pulses issued at a first frequency, wherein N equals two or greater, wherein the packets are issued at a second frequency;
receive a first measurement concerning a first symptom of the patient to assess the effect of the stimulation on the first symptom;
receive a second measurement concerning a second symptom of the patient to assess the effect of the stimulation on the second symptom;
in response to the first measurement, adjust the stimulation by adjusting either or both of (i) the first frequency of the pulses in each packet, or (ii) the number N of pulses provided in each packet; and
in response to the second measurement, adjust the stimulation by adjusting the second frequency at which the packets are issued.
